(19) United States
(12) Patent Application Publication
(10) Pub. No.: US 2025/0237799 A1
(43) Pub. Date: Jul. 24, 2025

(54) LIGHT SOURCE DEVICE AND SYSTEM

(71) Applicant: FUJIFILM Corporation, Tokyo (JP)

(72) Inventors: Takeshi FUKUDA, Ashigarakami-gun (JP); Masato YOSHIOKA, Ashigarakami-gun (JP); Makoto SUGIZAKI, Ashigarakami-gun (JP)

(73) Assignee: FUJIFILM Corporation, Tokyo (JP)

(21) Appl. No.: 19/023,310

(22) Filed: Jan. 16, 2025

(30) Foreign Application Priority Data
Jan. 19, 2024 (JP) 2024-006827

(51) Int. Cl. F21V 8/00; A61B 1/06

(52) U.S. Cl. CPC G02B 6/0006; A61B 1/0638; A61B 1/0646; A61B 1/0655; A61B 1/0661

(57) ABSTRACT

A light source device includes: five or more light sources that generate light of different colors; and an optical member that is configured to introduce the light generated by the five or more light sources into a light guide of an endoscope. A specific light source excluding four light sources among the five or more light sources has a wavelength of the generated light longer than a wavelength of light generated by the four light sources, and the light source device further includes a first light detection element that includes silicon and detects a part of the light generated by the specific light source, and a light reducing member that reduces light incident on the first light detection element.

### CROSS-REFERENCE TO RELATED APPLICATIONS

This application is based on and claims priority under 35 USC 119 from Japanese Patent Application No. 2024-006827 filed on Jan. 19, 2024, the contents of which are incorporated herein by reference.

### BACKGROUND OF THE INVENTION

### 1. Field of the Invention

The technology of the present disclosure relates to a light source device and a system.

### 2. Description of the Related Art

CN112904549A, CN115227188A, CN115227187A, and CN115200703A disclose a light source device of an endoscope.

CN115296744C, CN218899384Y, and CN215838929Y disclose a technique related to a connection between an endoscope and a control device.

### SUMMARY OF THE INVENTION

The technology of the present disclosure provides a light source device and a system of an endoscope that can control light emitted from an endoscope with high accuracy.

A light source device according to an aspect of the technology of the present disclosure comprises five or more light sources that generate light of different colors; and an optical member that is configured to introduce the light generated by the five or more light sources into a light guide of an endoscope, in which a specific light source excluding four light sources among the five or more light sources has a wavelength of the generated light longer than a wavelength of light generated by the four light sources, and the light source device includes a first light detection element that is composed of silicon and detects a part of the light generated by the specific light source, and a light reducing member that reduces light incident on the first light detection element.

A system according to an aspect of the technology of the present disclosure comprises the light source device and a processor, in which the processor controls the amounts of light generated by at least two of the five or more light sources.

According to the technology of the present disclosure, it is possible to control the light emitted from the endoscope with high accuracy.

### BRIEF DESCRIPTION OF THE DRAWINGS

FIG. 1 is an external view showing an endoscope apparatus according to an aspect of the technology of the present disclosure.

FIG. 2 is a block diagram showing a configuration of the endoscope apparatus 2 of FIG. 1.

FIG. 3 is a schematic view showing an example of an appearance of the endoscope connector 18.

FIG. 4 is a schematic view showing an example of an internal configuration of a light source unit 59 shown in FIG. 2.

FIG. 5 is a view showing a first modification example of the light source unit 59.

FIG. 6 is a view showing a second modification example of the light source unit 59.

FIG. 7 is a schematic cross-sectional view of a spectral filter 92 and a two-color light detection element 76 of the light source unit 59 shown in FIG. 6.

FIG. 8 is a schematic view showing an example of a hardware configuration for an endoscope 10 and a control device 11 to communicate with each other.

### DESCRIPTION OF THE PREFERRED EMBODIMENTS

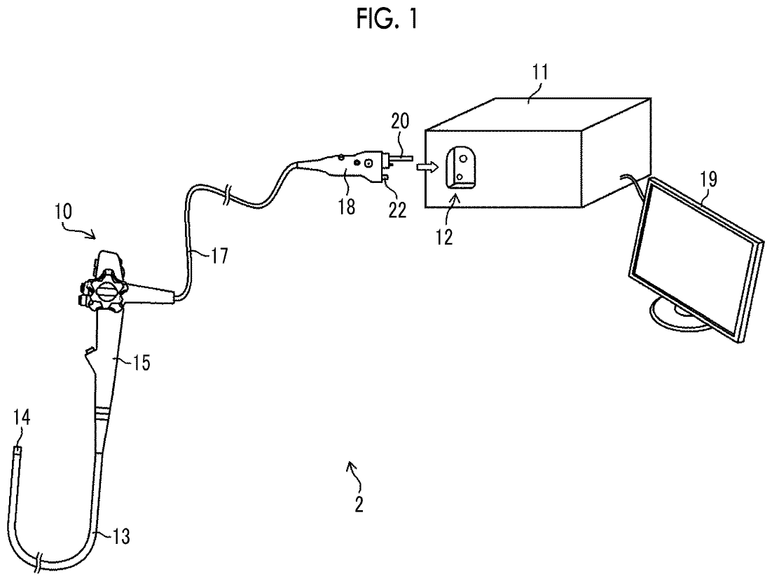

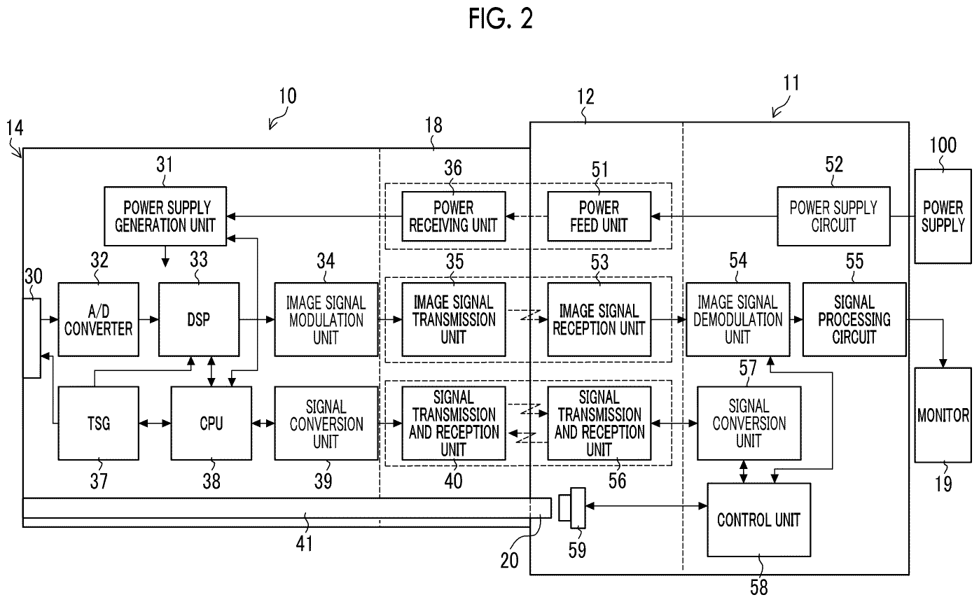

FIG. 1 is an external view showing an endoscope apparatus according to an aspect of the technology of the present disclosure. FIG. 2 is a block diagram showing a configuration of the endoscope apparatus 2 of FIG. 1.

As shown in FIG. 1, the endoscope apparatus 2 comprises an endoscope 10, a control device 11, and a monitor 19. The control device 11 generally controls the entire endoscope apparatus 2 in accordance with an operation of an operator input from an input device (not shown) (an operation switch, a keyboard, a mouse, and the like).

The endoscope 10 is an example of a flexible endoscope, and includes a flexible insertion part 13 to be inserted into a body cavity of a patient, an operation part 15 provided at a proximal end portion of the insertion part 13, a universal cord 17 provided at the operation part 15, and an endoscope connector 18 provided at an end part of the universal cord 17 and connected to the connector 12 of the control device 11. The endoscope 10 is not limited to a flexible endoscope, and may be another type of endoscope such as a rigid endoscope.

An observation window, an illumination window, and the like are provided on a distal end surface of the insertion part 13. A distal end part 14 that constitutes a distal end of the insertion part 13 is provided with an imaging unit 30 (see FIG. 2) including an objective optical system that forms an optical image of subject light from a part to be observed taken in through the observation window, and an imaging element that converts the optical image formed by the objective optical system into an image signal. The imaging element is, for example, a charge coupled device (CCD) image sensor, a complementary metal oxide semiconductor (CMOS) image sensor, or the like.

The image signal output from the imaging unit 30 is transmitted to the endoscope connector 18 by a transmission cable inserted into and disposed in the endoscope connector 18 through the inside of the insertion part 13, the operation part 15, and the universal cord 17.

A light emitting unit of a light guide 41 that transmits light to be emitted to the part to be observed from the illumination window is disposed in the distal end part 14. The light guide 41 is inserted into and disposed in the endoscope connector 18 through the inside of the insertion part 13, the operation part 15, and the universal cord 17. The light guide rod 20 connected to the light guide 41 is provided to protrude from the endoscope connector 18.

The operation part 15 includes an angle knob for adjusting the orientation of the distal end surface of the insertion part 13 in the vertical and horizontal directions, an air/water supply button for ejecting air and water from the distal end surface of the insertion part 13, a release button for recording a still image of the captured image, and the like. The orientation of the distal end surface of the insertion part 13 is adjusted by bending a bendable part provided in the vicinity of the proximal end side of the distal end part 14.

The universal cord 17 is covered with an outer wall part that is tubular and elongated, and has flexibility. The transmission cable and the light guide 41 described above, which are inserted into and disposed in cavity parts inside the insertion part 13 and inside the operation part 15, the air/water supply tube, and the like are inserted into and disposed in a pipe inside the outer wall part.

The endoscope connector 18 is connected to the connector 12 of the control device 11. The endoscope connector 18 and the connector 12 perform the supply of power from the control device 11 to the endoscope 10, the transmission of the image signal from the endoscope 10 to the control device 11, and the transmission and reception of the control signal between the endoscope 10 and the control device 11 in a noncontact manner without the physical connection of the electrical wires.

As shown in FIG. 2, the endoscope 10 includes a power receiving unit 36 provided in the endoscope connector 18. The control device 11 includes a power feed unit 51 provided in the connector 12. Power required for driving the internal circuit of the endoscope 10 is supplied from the control device 11 by a noncontact power supply means consisting of the power feed unit 51 of the control device 11 and the power receiving unit 36 of the endoscope 10.

The noncontact power supply means is a means for transmitting and receiving power in a noncontact manner by using electromagnetic coupling. In a case where the endoscope connector 18 is mounted on the connector 12, the power feed unit 51 and the power receiving unit 36 are disposed close to each other at a distance at which electromagnetic coupling is possible, and a state is set in which power transmission from the power feed unit 51 to the power receiving unit 36 is possible in a noncontact manner. The power feed unit 51 is connected to a commercial power supply 100 outside the control device 11 via a power supply circuit 52. Power supplied from the commercial power supply 100 and generated by the power supply circuit 52 is supplied to the power feed unit 51. Power is supplied from the power feed unit 51 to the power receiving unit 36 in a noncontact manner by the power supplied from the power supply circuit 52 to the power feed unit 51. The power receiving unit 36 receives power from the power feed unit 51 in a noncontact manner.

It is preferable that the power feed unit 51 is a primary coil connected to the power supply circuit 52 and the power receiving unit 36 is a secondary coil electromagnetically coupled to the primary coil. Examples of the structure of the primary coil and the secondary coil include a structure having a substrate having a flat surface and a coil spirally wound on the flat surface.

The endoscope 10 includes an image signal transmission unit 35 provided in the endoscope connector 18, a signal transmission and reception unit 40 provided in the endoscope connector 18, a power supply generation unit 31 that generates a power supply voltage to be supplied to each unit of the endoscope 10 from power received by the power receiving unit 36, an analog/digital (A/D) converter 32, a digital signal processor (DSP) 33, an image signal modulation unit 34, a timing signal generator (TSG) 37, a central processing unit (CPU) 38 that controls each unit of the endoscope 10, and a signal conversion unit 39.

The image signal output from the imaging unit 30 is changed from an analog signal to a digital signal by the A/D converter 32. The image signal output from the A/D converter 32 is transmitted to the DSP 33. The DSP 33 performs necessary processing such as amplification, gamma correction, and white balance processing on the image signal from the A/D converter 32.

The image signal modulation unit 34 performs optical modulation based on the image signal to generate an image light signal in order to transmit the image signal from the DSP 33 to the control device 11 by optical communication.

The image signal transmission unit 35 irradiates the control device 11 with light (light based on the image signal) in accordance with the image light signal generated by the image signal modulation unit 34. The image signal transmission unit 35 may be a light emitting device that can emit light for optical communication, and examples thereof include a laser light emitting element and a light emitting diode. The laser light emitting element refers to an element that emits laser light, which is coherent light, and examples thereof include a gas laser, a solid-state laser, and a semiconductor laser.

The signal conversion unit 39 performs optical modulation based on the control signal and demodulation of the control signal in order to transmit and receive the control signal that needs to be transmitted and received between the endoscope 10 and the control device 11 via optical communication. The control signal includes a first control signal transmitted from the control device 11 to the endoscope 10 in order to control the imaging unit 30 and the like, and a second control signal transmitted from the endoscope 10 to the control device 11.

The signal conversion unit 39 performs optical modulation based on the second control signal output from the CPU 38 to generate a second control light signal. The signal conversion unit 39 demodulates the electric signal output from the signal transmission and reception unit 40 to obtain a first control signal.

The signal transmission and reception unit 40 includes a light emitting device that irradiates the control device 11 with light (light based on the second control signal) in accordance with the second control light signal generated by the signal conversion unit 39, and a light-receiving device that receives light (light based on the first control signal) in accordance with the first control light signal modulated based on the first control signal generated by the control device 11.

Examples of the light emitting device of the signal transmission and reception unit 40 include a laser light emitting element and a light emitting diode. Examples of the light-receiving device of the signal transmission and reception unit 40 include a light-receiving element such as a semiconductor device such as a photodiode or a phototransistor.

The control device 11 includes an image signal reception unit 53 provided in the connector 12, a signal transmission and reception unit 56 provided in the connector 12, an image signal demodulation unit 54, a signal processing circuit 55, a signal conversion unit 57, and a control unit 58.

The image signal reception unit 53 is a light-receiving device that receives light emitted from the image signal transmission unit 35 and converts the light into an electric signal, and examples thereof include a light-receiving element such as a semiconductor device such as a photodiode or a phototransistor.

The image signal demodulation unit 54 demodulates the image signal transmitted from the endoscope 10 based on the electric signal output from the image signal reception unit 53. The image signal is processed by the signal processing circuit 55 and output to the monitor 19. In addition, the image signal is transmitted to the control unit 58 and is used to generate a control signal for adjusting the amount of light by a so-called auto exposure (AE) function or the like, or is processed to generate an endoscope image, and the endoscope image is recorded in a memory or the like.

The signal conversion unit 57 performs optical modulation based on the first control signal output from the control unit 58 to generate a first control light signal. The signal conversion unit 57 demodulates the electric signal output from the signal transmission and reception unit 56 to obtain a second control signal.

The signal transmission and reception unit 56 includes a light emitting device that irradiates the light-receiving device of the signal transmission and reception unit 40 of the endoscope 10 with light (light based on the first control signal) in accordance with the first control light signal generated by the signal conversion unit 57, and a light-receiving device that receives the light emitted from the light emitting device of the signal transmission and reception unit 40 of the endoscope 10.

Examples of the light emitting device of the signal transmission and reception unit 56 include a laser light emitting element and a light emitting diode. Examples of the light-receiving device of the signal transmission and reception unit 56 include a light-receiving element such as a semiconductor device such as a photodiode or a phototransistor.

In a case where the endoscope connector 18 is mounted on the connector 12 of the control device 11, the image signal transmission unit 35 and the image signal reception unit 53 are disposed close to each other at a distance at which optical communication can be performed, and a state is set in which the image signal transmission unit 35 and the image signal reception unit 53 can perform optical communication in a noncontact manner. In addition, the signal transmission and reception unit 40 and the signal transmission and reception unit 56 are disposed close to each other at a distance at which optical communication can be performed, and a state is set in which noncontact optical communication can be performed between the signal transmission and reception unit 40 and the signal transmission and reception unit 56. As described above, in the present embodiment, the communication of the image signal and the control signal between the endoscope 10 and the control device 11 is performed by the noncontact optical communication means. A method of optical communication is not particularly limited, and various methods can be employed. Wireless communication or magnetic communication can also be used instead of the optical communication.

The control device 11 includes a light source unit 59. The light source unit 59 has, for example, a light source including a xenon lamp, a semiconductor device such as a laser diode or a light emitting diode. In a case where the endoscope connector 18 is mounted on the connector 12 of the control device 11, the light guide rod 20 of the endoscope 10 is connected to the connector 12, and the light emitting unit of the light source unit 59 and the light guide rod 20 are aligned with each other. As a result, the light from the light source unit 59 is transmitted to the distal end part 14 through the light guide rod 20 and the light guide 41.

The control unit 58 is mainly configured by a processor, controls each unit of the control device 11, and sends a control signal to the CPU 38 and the like that constitute an internal circuit of the endoscope 10 to control the entire endoscope apparatus 2.

The processor of control unit 58 is a central processing unit (CPU) which is a general-purpose processor that executes software to perform various functions, a programmable logic device (PLD) which is a processor whose circuit configuration is changeable after manufacturing such as a field programmable gate array (FPGA), a dedicated electric circuit which is a processor having a circuit configuration exclusively designed to execute specific processing such as an application specific integrated circuit (ASIC), or the like.

The processor may be configured of one processor or a combination of two or more processors having the same type or different types (for example, a plurality of FPGAs, or a combination of CPU and FPGA). More specifically, the hardware structure of the processor is an electrical circuit (circuitry) in which circuit elements, such as semiconductor elements, are combined. The control unit 58 may be provided with a processor that controls the light source unit 59 and a processor that controls a control unit including each unit other than the light source unit 59, respectively.

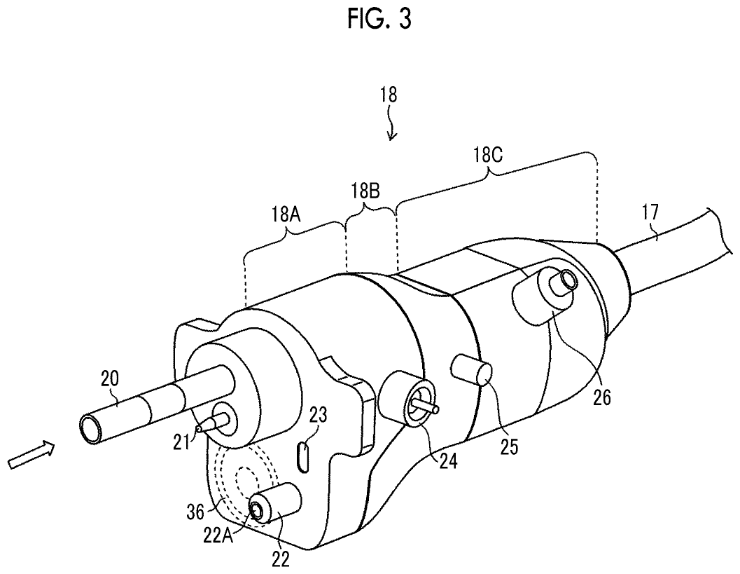

FIG. 3 is a schematic view showing an example of the appearance of the endoscope connector 18. The endoscope connector 18 includes a light guide rod 20 that protrudes from the endoscope connector 18 toward the connector 12 (not shown) and an image signal transmission connector 22.

The endoscope connector 18 can be configured with, for example, a first connector case 18A, a second connector case 18B, and a third connector case 18C in order from a side connected to the connector 12 of the control device 11.

The light guide rod 20 protrudes toward the connector 12 from the first connector case 18A having a connection surface to the connector 12. An air supply mouthpiece 21 is provided below the light guide rod 20 substantially parallel to the light guide rod 20. The air supply mouthpiece 21 communicates with an air and water supply pipe line provided in the endoscope 10 to supply air and water to the distal end part 14 of the endoscope 10.

The image signal transmission connector 22 protrudes from the connection surface with the connector 12 in the first connector case 18A in an insertion direction toward the connector 12. The image signal transmission connector 22 is used for aligning the image signal transmission unit 35 of the endoscope 10 with the image signal reception unit 53 of the control device 11. In particular, the image signal transmission unit 35 is disposed in an extension direction of a central axis of the image signal transmission connector 22. A window 22A is provided at a distal end of the image signal transmission connector 22 in order to transmit light. Light passes through the window 22A, and transmission and reception of light based on the image signal are performed between the image signal transmission unit 35 and the image signal reception unit 53.

A window 23 is provided at a position corresponding to the signal transmission and reception unit 40 on the connection surface with the connector 12 in the first connector case 18A. Through the window 23, transmission and reception of light based on the control signal are performed between the signal transmission and reception unit 40 and the signal transmission and reception unit 56.

The power receiving unit 36 is disposed inside the first connector case 18A and at a position close to the connection surface with the connector 12 in the first connector case 18A. Since the power receiving unit 36 is disposed inside the first connector case 18A and is not exposed to the outside.

An air/water supply connector 24 is provided on a side surface of the first connector case 18A. The air/water supply connector 24 is connected to a water supply tank (not shown). By operating an air/water supply button of the operation part 15, air or water can be supplied to the distal end part 14. The water supplied to the distal end part 14 removes the dirt on the lens surface of the distal end part 14. In addition, the air supplied to the distal end part 14 expands the lumen of the patient or removes water droplets on the lens.

For example, a balloon connector 25 is provided on a side surface of the second connector case 18B. By connecting the tube to the balloon connector 25, a balloon (not shown) provided in the insertion part 13 can be expanded and contracted. In a case of the endoscope 10 in which the balloon is not provided in the insertion part 13, it is not necessary to provide the balloon connector 25 in the endoscope connector 18.

A ventilation connector 26 is provided on a side surface of the third connector case 18C. The ventilation connector 26 enables a leak test for examining air leakage of the insertion part 13. The ventilation connector 26 communicates with the inside of the endoscope connector 18. Since the inside of the endoscope connector 18 communicates with the inside of each of the universal cord 17, the operation part 15, and the insertion part 13, the ventilation connector 26 communicates with the inside of the insertion part 13.

The universal cord 17 protrudes from an end part of the third connector case 18C.

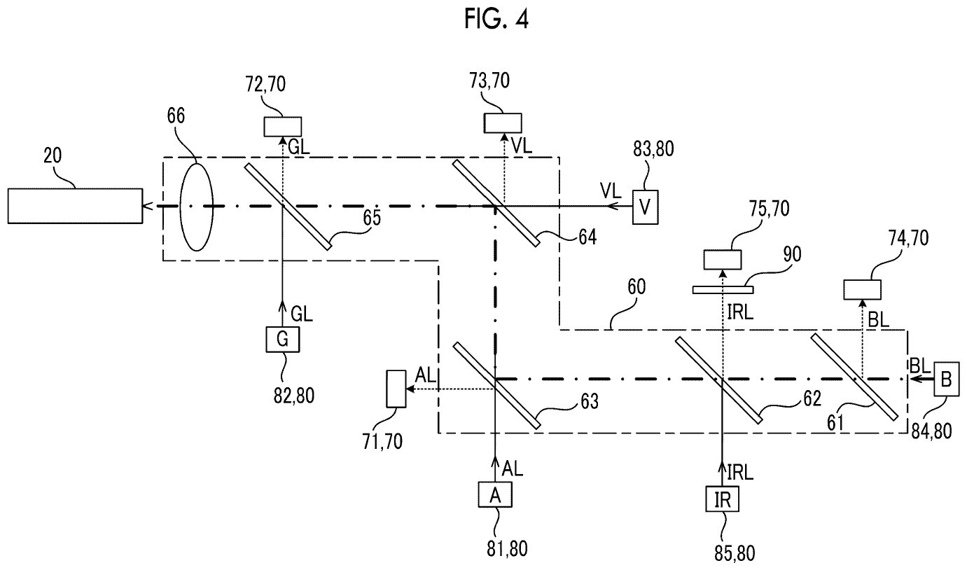

FIG. 4 is a schematic view showing an example of an internal configuration of the light source unit 59 shown in FIG. 2.

The light source unit 59 is an aspect of a light source device, and includes five light sources 80 that generate light of different colors, an optical member 60 that is configured to introduce the light generated by the five light sources 80 into the light guide 41 of the endoscope 10, five light detection elements 70 that are configured to detect a part of the light generated by each of the five light sources 80, and a light reducing member 90. The five light sources 80 are controlled by the control unit 58. The control unit 58 controls the amount of light of at least one of the five light sources 80 based on the light detected by the light detection element 70.

The five light sources 80 generate light in five wavelength ranges having different central wavelengths (wavelengths at which the intensity is maximum). The five light sources 80 include, for example, a B light source 84 that generates light in a blue wavelength range (hereinafter, referred to as B light), a V light source 83 that generates light in a violet wavelength range (hereinafter, referred to as V light), a G light source 82 that generates light in a green wavelength range (hereinafter, referred to as G light), an A light source 81 that generates light in an amber (or red) wavelength range (hereinafter, referred to as A light), and an IR light source 85 that generates light in a wavelength range (infrared wavelength range; infrared range) (hereinafter, referred to as IR light) having a central wavelength longer than a visible light range which is the combination of the four wavelength ranges.

In FIG. 4, the B light is denoted by BL, the V light is denoted by VL, the G light is denoted by GL, the A light is denoted by AL, and the IR light is denoted by IRL. Each light source 80 includes a light emitting element such as a light emitting diode or a laser diode, and is configured to include a phosphor, an excitation light cut filter, and the like as necessary. In FIG. 4, a thick one-dot chain line indicates a passing path of light generated by each light source 80 and incident into the light guide rod 20. In the five light sources 80, the central wavelengths of the light generated by the remaining IR light sources 85 excluding the four light sources 80, that is, the A light source 81, the G light source 82, the B light source 84, and the V light source 83, are longer than those of the four light sources 80. The IR light source 85 constitutes a specific light source. Light in the infrared range is used, for example, in a case of observing a submucosal layer of a subject part using a coloring agent such as indocyanine green (ICG) in endoscopy. In infra red imaging (IRI) performed using ICG, infrared light in a wavelength range of, for example, about 805 nm and about 940 nm is used in accordance with the absorption characteristics of ICG. In order to realize the IRI observation, the IR light source 85 may be provided with two light sources, one of which generates light in a wavelength range having a central wavelength of 805 nm and the other of which generates light in a wavelength range having a central wavelength of 940 nm.

The light detection element 70 is configured to include a light-receiving element such as a photodiode or a photoresistor. The five light detection elements 70 include a B light detection element 74 that is capable of detecting a part of the B light generated by the B light source 84, a V light detection element 73 that is capable of detecting a part of the V light generated by the V light source 83, a G light detection element 72 that is capable of detecting a part of the G light generated by the G light source 82, an A light detection element 71 that is capable of detecting a part of the A light generated by the A light source 81, and an IR light detection element 75 that is capable of detecting a part of the IR light generated by the IR light source 85. The IR light detection element 75 constitutes a first light detection element. The B light detection element 74, the V light detection element 73, the G light detection element 72, and the A light detection element 71 each constitute a second light detection element.

Since the five light detection elements 70 are each formed of the same semiconductor material, the manufacturing cost of the light source unit 59 can be reduced. It is preferable that the semiconductor material constituting the photoelectric conversion element included in the light detection element 70 is silicon. The IR light detection element 75 that detects the IR light can also be composed of a semiconductor material other than silicon, such as indium gallium arsenide (InGaAs). However, the InGaAs has high sensitivity in a wavelength range of 950 nm to 1700 nm. On the other hand, by using silicon, sensitivity in an infrared range in a wavelength range of 700 nm or more and 950 nm or less, which is necessary for IRI observation or the like, can also be increased.

The optical member 60 includes, for example, a dichroic mirror 65, a dichroic mirror 64, a dichroic mirror 63, a dichroic mirror 62, and a dichroic mirror 61 that are disposed in this order from the light guide rod 20 side along the above-described passing path, and a condenser lens 66 that is disposed between the dichroic mirror 65 and the light guide rod 20.

The B light generated by the B light source 84 is incident on the dichroic mirror 61. The B light incident on the dichroic mirror 61 transmits through the dichroic mirror 61 and is incident on the dichroic mirror 62, and a part of the B light is reflected to be incident on the B light detection element 74. The B light incident on the dichroic mirror 62 transmits through the dichroic mirror 62, is reflected by the dichroic mirror 63 to be incident into the dichroic mirror 64, is reflected by the dichroic mirror 64 to be incident into the dichroic mirror 65, transmits through the dichroic mirror 65, and reaches the light guide rod 20 via the condenser lens 66.

The IR light generated by the IR light source 85 is incident on the dichroic mirror 62. The IR light incident on the dichroic mirror 62 is reflected by the dichroic mirror 62 and is incident on the dichroic mirror 63, and a part of the IR light is transmitted through and is incident on the IR light detection element 75. The IR light incident on the dichroic mirror 63 is reflected by the dichroic mirror 63 to be incident into the dichroic mirror 64, is reflected by the dichroic mirror 64 to be incident into the dichroic mirror 65, transmits through the dichroic mirror 65, and reaches the light guide rod 20 via the condenser lens 66.

The A light generated by the A light source 81 is incident on the dichroic mirror 63. The A light incident on the dichroic mirror 63 transmits through the dichroic mirror 63, and a part of the A light is reflected to be incident on the A light detection element 71. The A light transmitted through the dichroic mirror 63 is incident on the dichroic mirror 64, is reflected by the dichroic mirror 64 to be incident into the dichroic mirror 65, is transmitted through the dichroic mirror 65, and reaches the light guide rod 20 via the condenser lens 66.

The V light generated by the V light source 83 is incident on the dichroic mirror 64. The V light incident on the dichroic mirror 64 transmits through the dichroic mirror 64, and a part of the V light is reflected to be incident into the V light detection element 73. The V light transmitted through the dichroic mirror 64 is incident on the dichroic mirror 65, is transmitted through the dichroic mirror 65, and reaches the light guide rod 20 via the condenser lens 66.

The G light generated by the G light source 82 is incident on the dichroic mirror 65. The G light incident on the dichroic mirror 65 is reflected by the dichroic mirror 65, reaches the light guide rod 20 via the condenser lens 66, and a part of the G light transmits through the dichroic mirror 65 and is incident on the G light detection element 72.

The position of each light source 80 is not limited to the position shown in FIG. 4 and can be optionally determined. In addition, the configuration of the optical member 60 is not limited to the configuration shown in FIG. 4. The optical member 60 can also employ other configurations as long as the optical member 60 is configured to introduce the A light into the light guide 41, introduce the B light into the light guide 41, introduce the V light into the light guide 41, introduce the G light into the light guide 41, and introduce the IR light into the light guide 41.

In addition, the light detection element 70 is provided at a position where light reflected by the dichroic mirror constituting the optical member 60 or light transmitted through the dichroic mirror is incident, but the present disclosure is not limited thereto. For example, the light detection element 70 may be provided at a position where another dichroic mirror is provided between the light source 80 and the dichroic mirror constituting the optical member 60 and the light reflected by this dichroic mirror is detected.

The light reducing member 90 is a member that can reduce the amount of light incident thereon, and is composed of, for example, a neutral density (ND) filter. The light reducing member 90 is provided between the IR light detection element 75 and the dichroic mirror 62. Therefore, the amount of IR light generated by the IR light source 85 and transmitted through the dichroic mirror 62 is reduced by the light reducing member 90 and is incident on the IR light detection element 75.

In a case where the photoelectric conversion element included in the IR light detection element 75 is composed of silicon, the light-receiving sensitivity of the IR light detection element 75 with respect to the wavelength range of the IR light is increased. In the present embodiment, a part of the IR light is incident on the IR light detection element 75 after the amount of the IR light is reduced by the light reducing member 90. Therefore, in the IR light detection element 75 having high light-receiving sensitivity to the IR light, it is possible to prevent the output from being saturated. As a result, the amount of light generated from the IR light source 85 can be accurately detected.

The light source unit 59 can be operated in a plurality of modes in which the number of light beams introduced into the light guide 41 of the endoscope 10 is different. The plurality of modes are selected according to the purpose in the endoscopy, and include, for example, a first mode in which A light, B light, G light, and V light are combined and introduced into the light guide 41, a second mode in which only IR light is introduced into the light guide 41, and a third mode in which G light and V light are combined and introduced into the light guide 41.

The control unit 58 controls the amounts of light generated by at least two light sources of the five or more light sources 80 based on the light detected by the light detection element 70. For example, in the first mode, the control unit 58 performs control such that the amounts of light of the A light, the B light, the G light, and the V light are the target values. In addition, the control unit 58 controls the ratio of amount of light of G light to V light in the third mode to be different from the ratio of amount of light of G light to V light in the first mode, based on the light detected by the G light detection element 72 and the V light detection element 73.

In the example of FIG. 4, the light detection element 70 that detects a part of the light generated by the light source 80 is provided in correspondence with each of the five light sources 80. However, the number of the light detection elements 70 may be smaller than the number of the light sources 80. The light detection element 70 may be provided only for the light source 80 for which the variable control of the amount of light is required. For example, in a case where the A light is always controlled to a constant amount of light, the A light detection element 71 may be omitted. As described above, depending on the control pattern of the light source 80, at least one of the B light detection element 74, the V light detection element 73, the G light detection element 72, or the A light detection element 71 can be omitted.

The light source unit 59 includes five light sources 80, but may have a configuration in which six or more light sources 80 that generate light of different colors are provided. In this case, the light source unit 59 may have a configuration in which four light sources 80 that generate light in the visible light range and two or more light sources 80 that generate light in the infrared range longer than the visible light range (for example, a light source that generates light having a central wavelength of 805 nm and a light source that generates light having a central wavelength of 940 nm) are provided as six or more light sources 80. As a result, it is possible to perform imaging using infrared light in different wavelength ranges. In a case where two or more light sources 80 that generate light in the infrared light range are provided, it is preferable to provide a light detection element 70 (a photoelectric conversion element is composed of silicon) that detects a part of light generated by each of the two or more light sources 80, and to provide a light reducing member 90 on a light incident side of the light detection element 70.

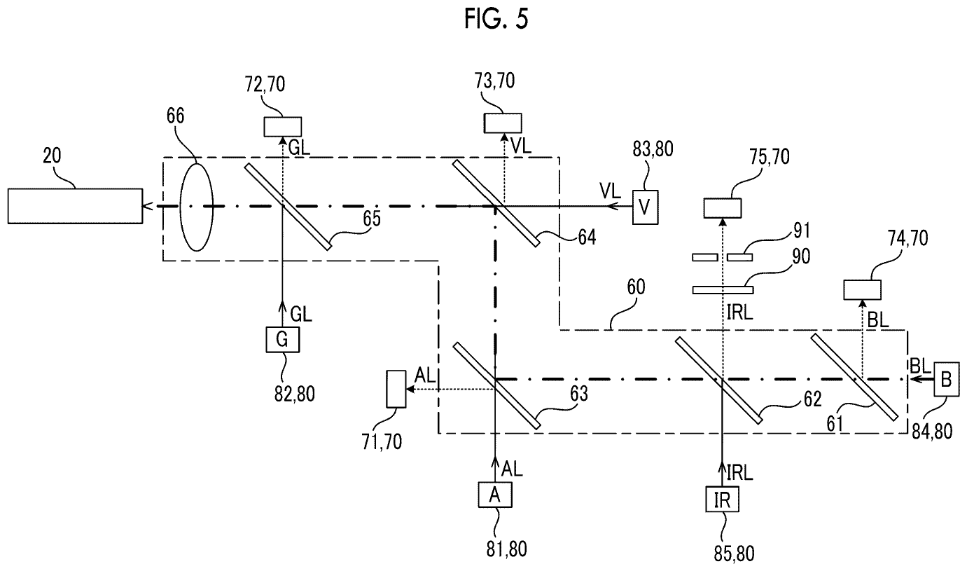

FIG. 5 is a view showing a first modification example of the light source unit 59. The light source unit 59 shown in FIG. 5 is the same as the light source unit 59 shown in FIG. 4 except that a restriction member 91 that restricts an incident range of light to the IR light detection element 75 is provided between the IR light detection element 75 and the light reducing member 90.

The restriction member 91 is configured with a slit, an aperture, or the like. Since the restriction member 91 is provided, the amount of light incident on the IR light detection element 75 is further reduced, and thus the saturation of the output of the IR light detection element 75 can be further prevented. Since the light reducing member 90 is present, it is not necessary to make the size of the slit or the aperture constituting the restriction member 91 minute. Therefore, the manufacturing cost of the light source unit 59 can be reduced. In addition, the restriction member 91 may be provided on the light incident side in the same manner for the light detection element 70 other than the IR light detection element 75.

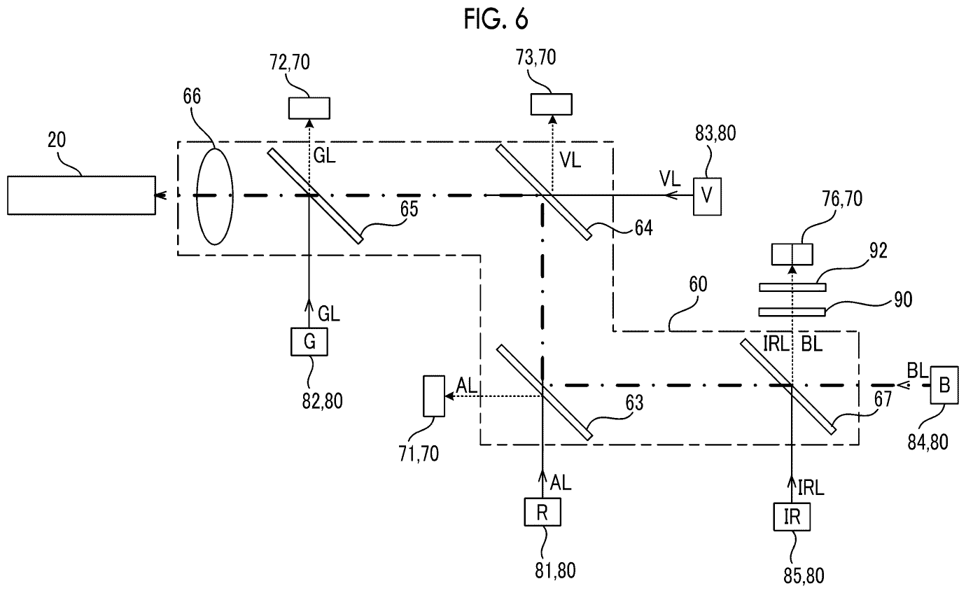

FIG. 6 is a view showing a second modification example of the light source unit 59. The light source unit 59 shown in FIG. 6 is the same as the light source unit 59 shown in FIG. 4 except that the dichroic mirror 61 and the B light detection element 74 are deleted, the dichroic mirror 62 is replaced with a dichroic mirror 67, the IR light detection element 75 is replaced with a two-color light detection element 76, and a spectral filter 92 is added.

The dichroic mirror 67 reflects a part of the B light generated by the B light source 84 to reach the two-color light detection element 76, and transmits the remaining B light to reach the dichroic mirror 63. The dichroic mirror 67 transmits a part of the IR light generated by the IR light source 85 to reach the two-color light detection element 76, and reflects the remaining IR light to reach the dichroic mirror 63.

In the two-color light detection element 76, a light-receiving region is divided into a plurality of regions, and an electric signal corresponding to the amount of received light is output in each light-receiving region. As the two-color light detection element 76, for example, a split type silicon photodiode manufactured by Hamamatsu Photonics K.K. is used.

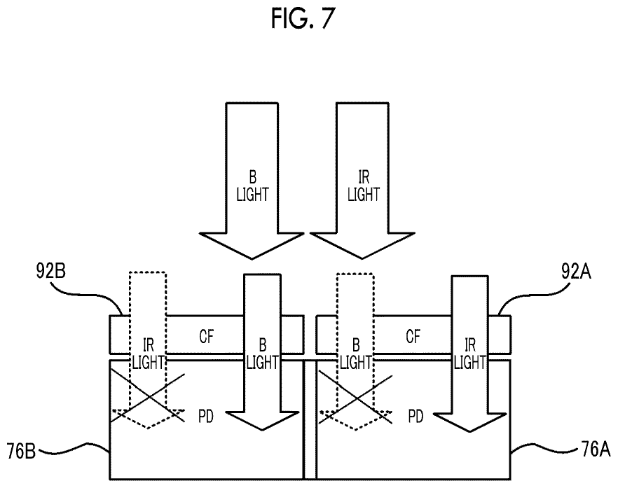

FIG. 7 is a schematic cross-sectional view of the spectral filter 92 and the two-color light detection element 76 of the light source unit 59 shown in FIG. 6. The two-color light detection element 76 includes a first light-receiving region 76A composed of silicon and a second light-receiving region 76B composed of silicon, and is capable of individually detecting light in each of the first light-receiving region 76A and the second light-receiving region 76B. The spectral filter 92 includes a first filter 92A provided on the light incident side of the first light-receiving region 76A and a second filter 92B provided on the light incident side of the second light-receiving region 76B.

The first filter 92A transmits the IR light generated by the IR light source 85 and absorbs or reflects the B light generated by the B light source 84. Therefore, only the IR light of the IR light and the B light transmitted through the light reducing member 90 can be incident on the first light-receiving region 76A.

The second filter 92B transmits the B light generated by the B light source 84 and absorbs or reflects the IR light generated by the IR light source 85. Therefore, only the B light of the IR light and the B light transmitted through the light reducing member 90 can be incident on the second light-receiving region 76B.

As shown in FIG. 6, by using the light detection element 70 in which the light-receiving regions are divided, it is possible to reduce the size and cost of the light source unit 59. The other light detection elements 70 other than the two-color light detection element 76 can also be configured with a split type light-receiving device including a plurality of light-receiving regions. In addition, the split type light-receiving device is not limited to a light-receiving device in which the light-receiving region is divided into two, and a light-receiving device in which the light-receiving region is divided into three or more can also be adopted. In the modification example shown in FIG. 6, the restriction member 91 shown in FIG. 5 can also be provided between the spectral filter 92 and the light reducing member 90.

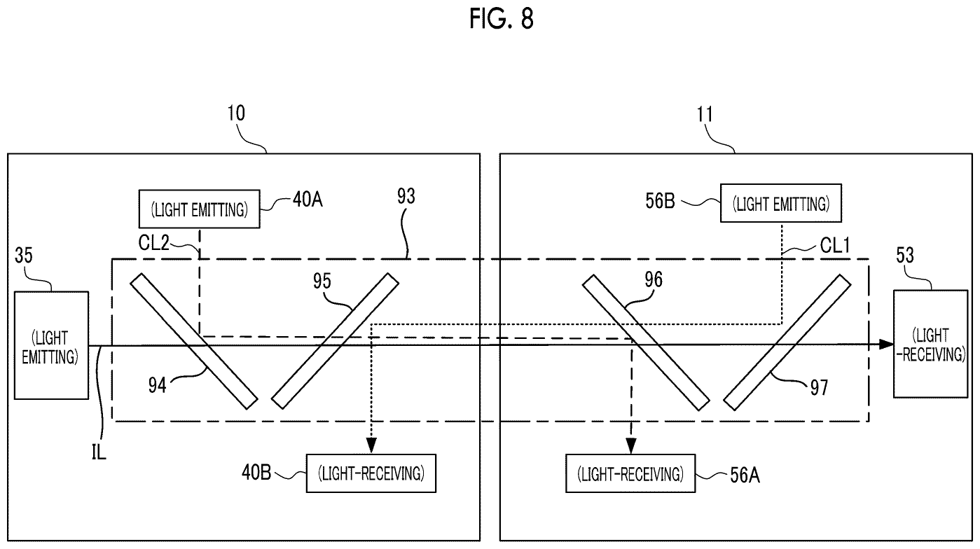

FIG. 8 is a schematic view showing and an example of a hardware configuration for the endoscope 10 and the control device 11 to communicate with each other. The endoscope 10 and the control device 11 are provided with an optical path 93 through which light used for communication passes. The optical path 93 may be separately provided for an optical path used for communication of the image signal and an optical path used for communication of the control signal. However, in the example of FIG. 8, two optical paths having different applications are unified into one optical path. That is, the optical path 93 serves as both an optical path of light based on the image signal and an optical path of light based on the control signals (the first control signal and the second control signal).

A dichroic mirror 94 and a dichroic mirror 95 are provided on the optical path 93 provided in the endoscope 10. A dichroic mirror 96 and a dichroic mirror 97 are provided on the optical path 93 provided in the control device 11.

The signal transmission and reception unit 40 of the endoscope 10 includes a light emitting device 40A and a light-receiving device 40B. The light IL based on the image signal emitted from the image signal transmission unit 35 is incident on the dichroic mirror 94, is transmitted through the dichroic mirror 94, is incident on the dichroic mirror 95, and is transmitted through the dichroic mirror 95 to the control device 11.

The light CL2 based on the second control signal emitted from the light emitting device 40A is incident on the dichroic mirror 94, is reflected by the dichroic mirror 94, is incident on the dichroic mirror 95, and is transmitted through the dichroic mirror 95 to the control device 11.

The signal transmission and reception unit 56 of the control device 11 includes a light emitting device 56B and a light-receiving device 56A. The light IL based on the image signal transmitted through the dichroic mirror 95 of the endoscope 10 is incident on the dichroic mirror 96, is transmitted through the dichroic mirror 96, is incident on the dichroic mirror 97, is transmitted through the dichroic mirror 97, and is incident on the image signal reception unit 53.

The light CL2 based on the second control signal transmitted through the dichroic mirror 95 of the endoscope 10 is incident on the dichroic mirror 96, is reflected from the dichroic mirror 96, and is incident on the light-receiving device 56A.

The light CL1 based on the first control signal emitted from the light emitting device 56B is incident on the dichroic mirror 97, is reflected from the dichroic mirror 97, is incident on the dichroic mirror 96, is transmitted through the dichroic mirror 96, and is incident on the dichroic mirror 95 of the endoscope 10. The light CL1 based on the first control signal incident on the dichroic mirror 95 is reflected from the dichroic mirror 95 and is incident on the light-receiving device 40B.

The optical path 93, the dichroic mirror 94, the dichroic mirror 95, the light emitting device 40A, the light-receiving device 40B, the image signal transmission unit 35, the image signal modulation unit 34, the signal conversion unit 39, the dichroic mirror 96, the dichroic mirror 97, the light-receiving device 56A, the light emitting device 56B, the image signal reception unit 53, the image signal demodulation unit 54, and the signal conversion unit 57 constitute a communication unit that performs optical communication of the image signal and the control signal between the endoscope 10 and the control device 11.

The image signal has a larger data amount than the control signal, and a high transfer rate is also required. Therefore, the communication frequency (modulation frequency) of the image signal is set to be higher than the communication frequency (modulation frequency) of the first control signal and the second control signal. In consideration of such a difference in communication frequency, it is preferable that the light emitting device of the image signal transmission unit 35 used for communication of the image signal and the light emitting device 40A and the light emitting device 56B used for communication of the control signal are different types.

For example, by using a light emitting device included in the image signal transmission unit 35 as a laser diode which is an example of a first type light emitting element, high-speed data transmission is facilitated. In addition, by using the light emitting diode, which is an example of a second type light emitting element, as the light emitting device 40A and the light emitting device 56B, it is possible to reduce the manufacturing cost. Since the image signals can be transferred at a high speed, responsiveness of the AE function based on the image signals transferred at a high speed can be improved. As a result, the amount of light control can be performed with higher accuracy by the amount of light control of the light source 80 and the AE function based on the output of the light detection element 70.

In the present embodiment, the endoscope apparatus 2 is an aspect of a system including the endoscope 10 and a control device 11 including a light source device. In addition, the control device 11 is an aspect of a system including the light source unit 59 and a processor (control unit 58).

As described above, at least the following matters are described in the present specification. In the following, the components corresponding to the above-described embodiments are shown in parentheses, but the present disclosure is not limited thereto.

(1)

A light source device including: five or more light sources that generate light of different colors; and an optical member that is configured to introduce the light generated by the five or more light sources into a light guide of an endoscope, in which a specific light source excluding four light sources among the five or more light sources has a wavelength of the generated light longer than a wavelength of light generated by the four light sources, and the light source device includes a first light detection element that is composed of silicon and detects a part of the light generated by the specific light source, and a light reducing member that reduces light incident on the first light detection element.

(2)

The light source device according to (1), in which the specific light source generates light in an infrared wavelength range.

(3)

The light source device according to (2), in which the light reducing member is a Neutral Density filter.

(4)

The light source device according to any one of (1) to (3), further including: a restriction member that is provided between the light reducing member and the first light detection element and that restricts an incident range of light to the first light detection element.

(5)

The light source device according to any one of (1) to (4), in which the first light detection element has a light-receiving region divided into a plurality of regions.

(6)

The light source device according to any one of (1) to (5), further including: a second light detection element that detects a part of light generated by at least one of the four light sources.

(7)

A system including: a control device including the light source device according to any one of (1) to (6); an endoscope that is connected to the control device; and a communication unit that is provided in the endoscope and the control device and that performs optical communication of an image signal and a control signal between the control device and the endoscope, in which the communication unit has an optical path through which light used for communication passes, the optical path serves as both an optical path of light based on the image signal and an optical path of light based on the control signal, and a communication frequency of the image signal is higher than a communication frequency of the control signal.

(8)

The system according to (7), in which the communication unit performs communication of the image signal using light generated by a first type light emitting element, and performs communication of the control signal using light generated by a second type light emitting element different from the first type light emitting element.

(9)

The system according to (8), in which the first type light emitting element is a laser diode, and the second type light emitting element is a light emitting diode.

(10)

A system including: the light source device according to any one of (1) to (6); and a processor, in which the processor controls amounts of light generated by at least two of the five or more light sources.

### EXPLANATION OF REFERENCES

IL, CL1, CL2: light
2: endoscope apparatus
10: endoscope
11: control device
12: connector
13: insertion part
14: distal end part
15: operation part
17: universal cord
18: endoscope connector
18A: first connector case
18B: second connector case
18C: third connector case
19: monitor
20: light guide rod
21: air supply mouthpiece
22: image signal transmission connector
22A, 23: window
24: air/water supply connector
25: balloon connector
26: ventilation connector
30: imaging unit
31: power supply generation unit
32: A/D converter
33: DSP
34: image signal modulation unit
35: image signal transmission unit
36: power receiving unit
37: timing signal generator
38: CPU
39, 57: signal conversion unit
40, 56: signal transmission and reception unit
40A, 56B: light emitting device
40B, 56A: light-receiving device
41: light guide
51: power feed unit
52: power supply circuit
53: image signal reception unit
54: image signal demodulation unit
55: signal processing circuit
58: control unit
59: light source unit
60: optical member
61, 62, 63, 64, 65, 67, 94, 95, 96, 97: dichroic mirror
66: condenser lens
70: light detection element
71: A light detection element
72: G light detection element
73: V light detection element
74: B light detection element
75: IR light detection element
76: two-color light detection element
76A: first light-receiving region
76B: second light-receiving region
80: light source
81: A light source
82: G light source
83: V light source
84: B light source
85: IR light source
90: light reducing member
91: restriction member
92: spectral filter
92A: first filter
92B: second filter
93: optical path
100: power supply

What is claimed is:

1. A light source device comprising:
five or more light sources that generate light of different colors; and
an optical member that is configured to introduce the light generated by the five or more light sources into a light guide of an endoscope,
wherein a specific light source excluding four light sources among the five or more light sources has a wavelength of the generated light longer than a wavelength of light generated by the four light sources, and
the light source device further includes
a first light detection element that includes silicon and detects a part of the light generated by the specific light source, and
a light reducing member that reduces light incident on the first light detection element.

2. The light source device according to claim 1,
wherein the specific light source generates light in an infrared wavelength range.

3. The light source device according to claim 2,
wherein the light reducing member is a Neutral Density filter.

4. The light source device according to claim 1, further comprising:
a restriction member that is provided between the light reducing member and the first light detection element and that restricts an incident range of light to the first light detection element.

5. The light source device according to claim 1,
wherein the first light detection element has a light-receiving region divided into a plurality of regions.

6. The light source device according to claim 1, further comprising:
a second light detection element that detects a part of light generated by at least one of the four light sources.

7. A system comprising:
a control device including the light source device according to claim 1;
an endoscope that is connected to the control device; and
a communication unit that is provided in the endoscope and the control device and that performs optical communication of an image signal and a control signal between the control device and the endoscope,
wherein the communication unit has an optical path through which light used for communication passes,
the optical path serves as both an optical path of light based on the image signal and an optical path of light based on the control signal, and
a communication frequency of the image signal is higher than a communication frequency of the control signal.

8. The system according to claim 7,
wherein the communication unit performs communication of the image signal using light generated by a first type light emitting element, and performs communication of the control signal using light generated by a second type light emitting element different from the first type light emitting element.

9. The system according to claim 8,
wherein the first type light emitting element is a laser diode, and
the second type light emitting element is a light emitting diode.

10. A system comprising:
the light source device according to claim 1; and
a processor,
wherein the processor controls amounts of light generated by at least two of the five or more light sources.
